(19) United States
(12) Patent Application Publication
(10) Pub. No.: US 2017/0268796 A1
(43) Pub. Date: Sep. 21, 2017

(54) INFORMATION PROCESSING SYSTEM, INFORMATION PROCESSING DEVICE, ACCLIMATIZATION INDICATOR DISPLAY DEVICE, AND METHOD FOR CONTROLLING INFORMATION PROCESSING SYSTEM

(71) Applicant: SEIKO EPSON CORPORATION, Tokyo (JP)

(72) Inventor: Eizo TAKAHASHI, Asahi-mura (JP)

(73) Assignee: SEIKO EPSON CORPORATION, Tokyo (JP)

(21) Appl. No.: 15/443,203

(22) Filed: Feb. 27, 2017

(30) Foreign Application Priority Data
Mar. 16, 2016 (JP) 2016-051967

(51) Int. Cl. F24F 11/00; A61B 5/145; G05B 15/02; A61B 5/00

(52) U.S. Cl. CPC F24F 11/0017; A61B 5/4809; A61B 5/14542; A61B 5/681; G05B 15/02; F24F 2011/0028; F24F 2011/0042; F24F 2011/0057

(57) ABSTRACT

An information processing system includes: a measuring unit which measures biological information of a user present in a closed space; and a processing unit which controls an environmental state of the closed space on the basis of the biological information. The processing unit controls a pressure which is an air pressure or partial pressure of oxygen in the closed space, or a concentration which is an oxygen concentration, on the basis of the biological information.

### CROSS-REFERENCES TO RELATED APPLICATIONS

This application claims priority to Japanese Patent Application No. 2016-051967, filed Mar. 16, 2016, the entirety of which is herein incorporated by reference.

### BACKGROUND

1. Technical Field

The present invention relates to an information processing system, an information processing device, an acclimatization indicator display device, and a method for controlling an information processing system.

2. Related Art

Long-distance athletes used to enhance their cardiopulmonary function by traveling to high-altitude places where air is thin and acclimatizing and adapting to high altitude. Recently, by using a system (hypoxic chamber) in which a closed space with increased airtightness and a device for controlling the air pressure within the closed space are provided, it is possible to achieve similar effects without actually traveling to high altitude.

For example, JP-A-2007-7171 discloses an environment simulation device which effectively associates biological information and the environment, using data from environment adjustment unit and a biological information monitoring unit.

In the case of actual high-altitude training or the like, since the athlete needs to travel from a relatively low-altitude place to a high-altitude place, it necessarily takes some time for the elevation above sea level to change. However, in the case of using an environment simulation device such as a hypoxic chamber as in JP-A-2007-7171, it is possible to change the environment in a relatively short time. Therefore, the time required for the environment change is short compared with the response time of the living body, and the user may be unable to properly acclimatize to the change in air pressure or oxygen concentration and therefore suffer health problems such as altitude sickness or decompression sickness.

Therefore, in the case of using a hypoxic chamber, an operator needs to be present in order to check the state of the trainee and secure the safety of the trainee. The operator needs to constantly monitor the trainee and this causes a heavy burden on the business operator and the operation manager who have introduced the hypoxic chamber and also on the operator. Moreover, in some cases, details of the control on the hypoxic chamber may be mainly determined subjectively by the operator. In such cases, an inappropriate determination or control may be carried out and there is also a risk of control failure due to human error.

### SUMMARY

An advantage of some aspects of the invention is that an information processing system, an information processing device, an acclimatization indicator display device, and a method for controlling an information processing system and the like can be provided in which the user is allowed to acclimatize properly, using biological information.

Another advantage of some aspects of the invention is that an information processing system, an information processing device, an acclimatization indicator display device, and a method for controlling an information processing system and the like can be provided in which proper control of a closed space according to the state of the user is carried out using biological information for environmental state control.

An aspect of the invention relates to an information processing system including: a measuring unit which measures biological information of a user present in a closed space; and a processing unit which controls an environmental state of the closed space on the basis of the biological information. The processing unit controls a pressure which is an air pressure or partial pressure of oxygen in the closed space, or a concentration which is an oxygen concentration in the closed space, on the basis of the biological information.

According to this configuration, the information processing system controls the pressure or oxygen concentration in the closed space on the basis of the biological information of the user. Thus, since the control can be carried out while the state of the user is checked, it is possible to restrain the application of an excessive load on the user and to allow the user to do activities in the closed space safely and efficiently.

In the aspect of the invention, the processing unit may change the pressure or the concentration in the closed space if a value of the biological information satisfies a predetermined control execution condition.

With this configuration, it is possible to perform control that does not apply an excessive load on the user.

In the aspect of the invention, the processing unit may perform control to change the pressure or the concentration in the closed space if the value of the biological information satisfies the control execution condition and the pressure or the concentration in the closed space does not reach a predetermined set value.

With this configuration, it is possible to perform control to cause the pressure or the concentration to reach a set value without applying an excessive load on the user.

In the aspect of the invention, the processing unit may perform control to maintain the pressure or the concentration in the closed space if the value of the biological information does not satisfy the control execution condition.

With this configuration, it is possible to perform control that does not apply an excessive load on the user.

In the aspect of the invention, the processing unit may find an acclimatization indicator of the user on the basis of the biological information of the user.

With this configuration, it is possible to find an indicator indicating the extent to which the user has acclimatized to change in the environment, on the basis of the biological information.

In the aspect of the invention, the processing unit may find, as an acclimatization indicator of the user, at least one of pressure information about the pressure in the case where the value of the biological information no longer satisfies the control execution condition, concentration information about the concentration in the case where the value of the biological information no longer satisfies the control execution condition, and time information about a timing when the value of the biological information satisfies the control execution condition after the pressure reaches the set value.

With this configuration, it is possible to find at least one of the pressure information, the concentration information, and the time information, as the acclimatization indicator.

In the aspect of the invention, the processing unit may find, as the pressure information which is the acclimatization indicator, at least one of a value of the pressure in the case where the value of the biological information no longer satisfies the control execution condition, and difference information indicating a change in the pressure over a period until the value of the biological information no longer satisfies the control execution condition from before the pressure begins to change.

With this configuration, it is possible to find the value of the pressure itself or the difference information, as the pressure information.

In the aspect of the invention, the processing unit may find, as the concentration information which is the acclimatization indicator, at least one of a value of the concentration in the case where the value of the biological information no longer satisfies the control execution condition, and difference information indicating a change in the concentration over a period until the value of the biological information no longer satisfies the control execution condition from before the concentration begins to change.

With this configuration, it is possible to find the value of the concentration itself or the difference information, as the concentration information.

In the aspect of the invention, the processing unit may find, as the time information which is the acclimatization indicator, at least one of first time information indicating a time period from a timing when the pressure or the concentration begins to change to a timing after the pressure or the concentration reaches the set value and when the value of the biological information satisfies the control execution condition, and second time information indicating a time period from a timing when the pressure or the concentration reaches the set value to a timing when the value of the biological information satisfies the control execution condition.

With this configuration, it is possible to find the time information on the basis of the time period from a predetermined timing to the timing when the value of the biological information satisfies the control execution condition.

In the aspect of the invention, the processing unit may determine whether to return the pressure or the concentration to a reference value or not, on the basis of the biological information in a sleeping state of the user.

With this configuration, it is possible to allow the user to safely execute activities involving sleep in the closed space.

In the aspect of the invention, the processing unit may determine whether to return the pressure or the concentration to a reference value or not, on the basis of the biological information after the pressure or the concentration in the closed space reaches the set value.

With this configuration, it is possible to allow the user to safely execute activities in the closed space where the pressure or the concentration has reached a set value.

In the aspect of the invention, the processing unit may perform control to return the pressure or the concentration to the reference value, if a number of times the value of the biological information satisfies a predetermined interruption condition reaches a predetermined number of times or above.

With this configuration, it is possible to perform control on the basis of the number of times the biological information satisfies the interruption condition.

In the aspect of the invention, the biological information may include arterial blood oxygen saturation information.

With this configuration, it is possible to use the arterial blood oxygen saturation information as the biological information.

Another aspect of the invention relates to an information processing system including: a measuring unit which measures biological information of a user present in a closed space; and a processing unit which controls an environmental state of the closed space on the basis of the biological information. The biological information includes arterial blood oxygen saturation information. The processing unit controls the environmental state of the closed space on the basis of the arterial blood oxygen saturation information.

According to the another aspect of the invention, the information processing system controls the environmental state of the closed space on the basis of the arterial blood oxygen saturation information, which is the biological information of the user. Thus, since the environmental state can be controlled while the state of the user is checked, it is possible to restrain the application of an excessive load on the user and to allow the user to do activities in the closed space safely and efficiently.

In the another aspect of the invention, the processing unit may perform control to change the environmental state of the closed space on condition that a value of the arterial blood oxygen saturation information is equal to or above a predetermined threshold.

With this configuration, it is possible to perform environmental state control which does not apply an excessive load on the user.

Another aspect of the invention relates to an information processing device including: an acquisition unit which acquires biological information of a user present in a closed space; and a processing unit which controls an environmental state of the closed space on the basis of the biological information. The biological information includes at least arterial blood oxygen saturation information. The processing unit controls the environmental state of the closed space, at least on the basis of the arterial blood oxygen saturation information.

According to the another aspect of the invention, the information processing device acquires the biological information of the user including the arterial blood oxygen saturation information and controls the environmental state of the closed space on the basis of the acquired biological information. Thus, since the environmental state can be controlled while the state of the user is checked, it is possible to restrain the application of an excessive load on the user and to allow the user to do activities in the closed space safely and efficiently.

In the another aspect of the invention, the processing unit may perform control to change the environmental state of the closed space if a value of the arterial blood oxygen saturation information is equal to or above a predetermined threshold.

With this configuration, it is possible to perform environmental state control which does not apply an excessive load on the user.

Another aspect of the invention relates to an acclimatization indicator display device including: an acquisition unit which acquires an acclimatization indicator indicating an extent of acclimatization of a user present in a closed space to an environmental state of the closed space on the basis of biological information including at least arterial blood oxygen saturation information of the user; and a display unit which displays the acclimatization indicator.

According to another aspect of the invention, the acclimatization indicator of the user to the environmental state of the closed space is acquired and displayed on the basis of the biological information of the user present in the closed space. Accordingly, the degree of acclimatization to the environmental state is acquired based on the biological information measured and can be present to the user as the acclimatization indicator.

Another aspect of the invention relates to a method for controlling an information processing system including: performing measurement processing of biological information of a user present in a closed space; and controlling a pressure which is an air pressure or partial pressure of oxygen in the closed space, or a concentration which is an oxygen concentration in the closed space, on the basis of the biological information.

Another aspect of the invention relates to a method for controlling an information processing system including: performing measurement processing of biological information including at least arterial blood oxygen saturation information of a user present in a closed space; and controlling an environmental state of the closed space on the basis of the arterial blood oxygen saturation information.

### BRIEF DESCRIPTION OF THE DRAWINGS

The invention will be described with reference to the accompanying drawings, wherein like numbers reference like elements.

FIG. 1 shows an example of the configuration of an information processing system.

FIG. 2 shows an example of the configuration of a system (environment chamber) of the information processing system.

FIG. 3 shows an example of the appearance of a wearable device.

FIG. 4 shows an example of the appearance of a wearable device.

FIG. 5 explains the principle of a technique for acquiring arterial blood oxygen saturation information.

FIG. 6 shows an example of a pressure control profile in the case where biological information is not used.

FIG. 7 shows a pressure control profile based on biological information.

FIG. 8 is a flowchart for explaining environmental state control according to an embodiment.

FIG. 9 is a flowchart for explaining forced halt control.

FIG. 10 shows an example of change of a pressure change acclimatization indicator due to the number of times the training is conducted.

FIG. 11 shows another example of the pressure control profile in the case where biological information is not used.

FIG. 12 shows another example of the pressure control profile based on biological information.

FIG. 13 is another flowchart for explaining the environmental state control according to the embodiment.

### DESCRIPTION OF EXEMPLARY EMBODIMENTS

Hereinafter, an embodiment will be described. The following embodiment should not unduly limit the contents of the invention described in the appended claims. Also, not all the configurations described in the embodiment are necessarily essential elements of the invention.

### 1. Technique According to Embodiment

First, a technique according to the embodiment will be described. An athlete of a sport where endurance is important, such as long-distance sports, may carry out altitude training in order to enhance cardiopulmonary function. Altitude training is to achieve enhancement of cardiopulmonary function by doing activities in a thin-air environment. Therefore, if a thin-air environment can be prepared, the athlete can expect similar training effects without actually traveling to high altitude.

Specifically, by using a hypoxic chamber including a closed space with high airtightness and a device for controlling the air pressure (in a narrow sense, partial pressure of oxygen) in the closed space, and carrying out activities in the closed space with its oxygen concentration reduced, the athlete can achieve effects similar to those of altitude training.

However, in the case where such a hypoxic chamber is used, there are problems that would not take place in the case of actually traveling to high altitude and carrying out training there. Specifically, oxygen concentration can change more quickly within a short period of time than the response speed of a living body to acclimatize to the change in oxygen concentration. Therefore, there is a risk that symptoms of altitude sickness such as headache, nausea, and sleep disorder may occur. In some cases, serious conditions such as cerebral edema and pulmonary edema may occur.

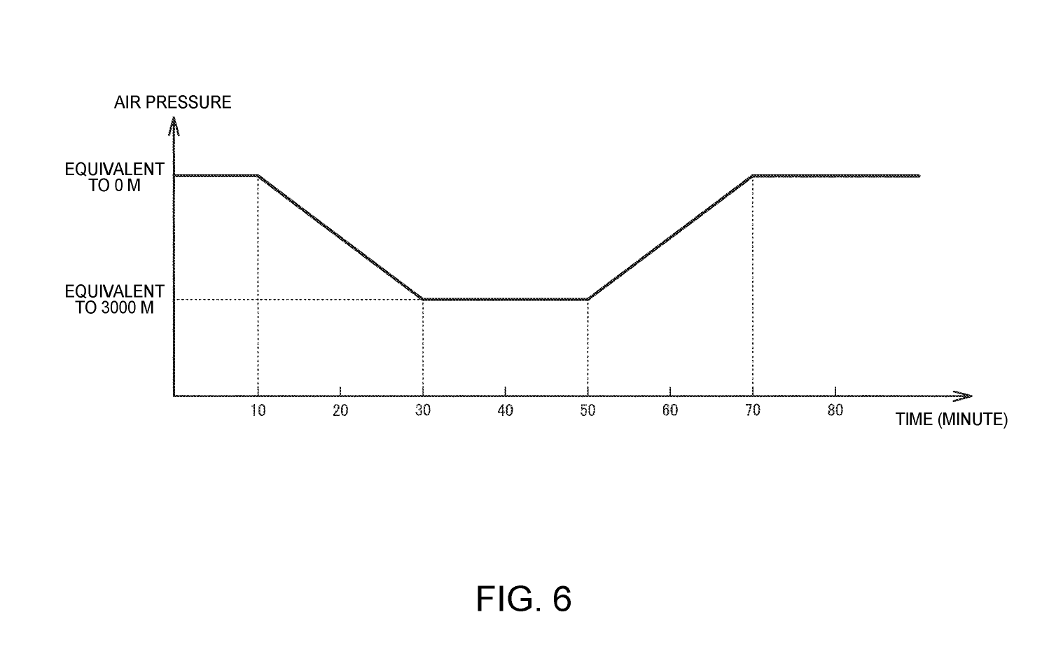

When a hypoxic chamber is used, a technique of changing the environment (in a narrow sense, air pressure) in the closed space according to a predetermined profile is conceivable, as described later with reference to FIG. 6, for example. However, the response to an environment where oxygen is low varies from one user to another. Even with the same profile, some users do not experience any abnormality whereas some users develop symptoms of altitude sickness. Therefore, simply controlling the environment according the profile is not equivalent to control taking user's safety into account.

Also, when using a hypoxic chamber, it is conceivable that an operator is deployed and made to monitor the state of the hypoxic chamber. The operator monitors the state such as air pressure in the hypoxic chamber and performs environmental control on the hypoxic chamber so that the user (hypoxic chamber user or trainee) can safely carry out activities. However, with the related-art technique, the operator subjectively determines control contents and therefore can make an inappropriate determination or control. Also, there is a risk of control error due to human error.

JP-A-2007-7171 discloses the association between environment data and biological data, but the same literature does not disclose any appropriate technique for controlling an environment simulation device which takes user's safety into account.

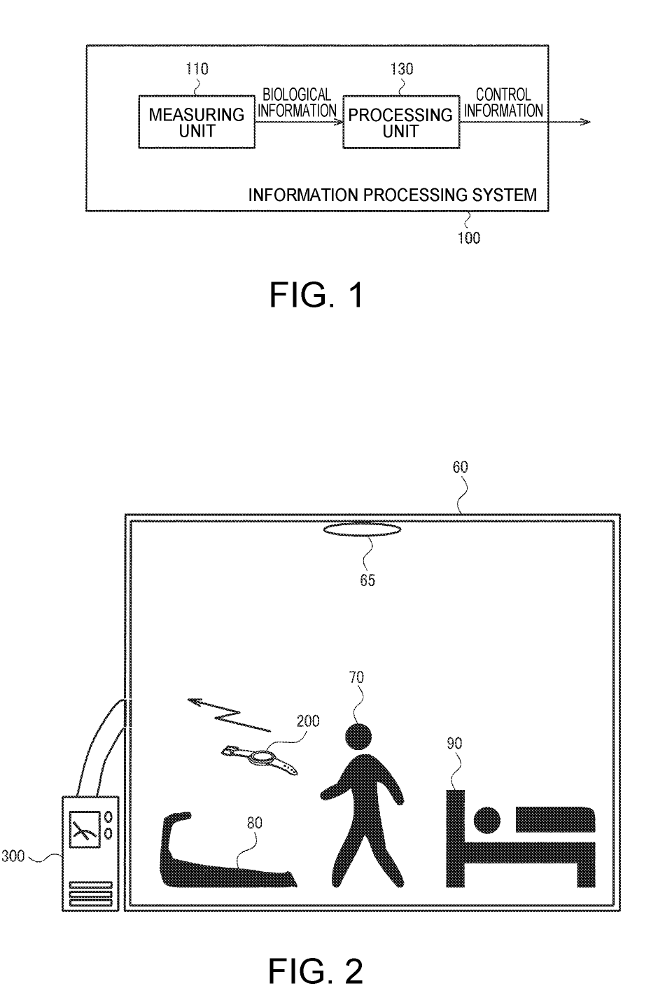

In view of the above, the applicant proposes a technique for controlling the environment of a closed space on the basis of biological information of a user. An information processing system 100 according to this embodiment includes a measuring unit 110 which measures biological information of a user present in a closed space, and a processing unit 130 which controls the environmental state of the closed space on the basis of the biological information, as shown in FIG. 1. The processing unit 130 controls a pressure which is the air pressure or partial pressure of oxygen in the closed space, or a concentration which is the oxygen concentration in the closed space, on the basis of the biological information.

Here, the closed space refers to a space that is closed so as to change its internal environment to an extent clearly distinguishable from the external environment. The member to be the boundary between the inside and outside of the closed space can be made up of various materials. A hard member such as a metal, or a soft member such as vinyl or cloth may be used.

The environmental state of the closed space includes the state of pressure, the state of oxygen concentration, the state of temperature, the state of humidity, the state of illuminance or the like in the closed space. In this example, since it is assumed that the pressure or concentration in the closed space is to be controlled, the closed space needs to be highly airtight. Meanwhile, if the processing unit 130 controls the temperature in the closed space, the closed space needs to be highly heat-insulative. If the humidity is to be controlled, the airtightness in the closed space may be made higher, as in the example of pressure control. If the illuminance is to be controlled, the member forming the closed space may be a member that does not easily transmit light.

The biological information is information indicating the state of biological activities of the user. In a narrow sense, the biological information in this embodiment may include arterial blood oxygen saturation information. The arterial blood oxygen saturation information is information indicating oxygen saturation (degree of bonding between hemoglobin and oxygen) in the blood in the artery. More specifically, the value of SpO2(%), which is percutaneous arterial blood oxygen saturation, may be used. However, the biological information is not limited to this example and may be pulse wave information such as pulse rate or pulse interval (RR interval) or may include information about respiration, body temperature, perspiration and the like.

In this way, the biological information of the user can be used for controlling the closed space. Since the biological information is information indicating the state of biological activities of the user as described above, if the user is under the influence of a change in the environmental state of the closed space, it is considered that the influence emerges as a change in the biological information. That is, whether the user suffers a health risk of altitude sickness or the like, or not, can be determined objectively at an appropriate timing without simply relying on the subjective determination by the operator or the user. Thus, by controlling the environmental state on the basis of the biological information, it is possible to realize proper control which takes user's safety into account.

Particularly in the case of changing the oxygen concentration in the closed space by air pressure control or concentration control, if a sufficient amount of oxygen cannot be supplied to each part of the body because of a reduction in oxygen concentration, this emerges as a reduction in the numerical value of SpO2. Therefore, in the case of controlling the pressure or concentration as the environmental state, more appropriate control is enabled by using arterial blood oxygen saturation information as the biological information.

However, as described above, the environment information and the biological information in the embodiment can be implemented with various modifications. For example, the technique in the embodiment can be applied to an information processing system including a measuring unit 110 which measures biological information of a user present in a closed space and a processing unit 130 which controls the environmental state of the closed space on the basis of the biological information, wherein the biological information includes arterial blood oxygen saturation information and wherein the processing unit 130 controls the environmental state of the closed space on the basis of the arterial blood oxygen saturation information.

This enables control of the environmental state based on the arterial blood oxygen saturation information. The arterial blood oxygen saturation information is an indicator value indicating oxygen supplied to each part of the user's body. If this indicator value drops, the risk of occurrence of symptoms of altitude sickness rises. That is, proper control which takes user's safety into account is enabled by using the arterial blood oxygen saturation information.

An example of the configuration of the information processing system 100 according to the embodiment will be described below. Also, as an example of a device used to measure biological information of the user, an example of the configuration of a wearable device 200 will be described as well. Then, a specific example of control in the case of changing pressure as the environmental state will be described. Moreover, a technique of finding an acclimatization indicator of the user to an environmental change on the basis of a series of activity results will be described. Finally, several modifications will be described.

### 2. Example of System Configuration

An example of the configuration of the information processing system 100 is as shown in FIG. 1. The information processing system 100 includes a measuring unit 110 and a processing unit 130.

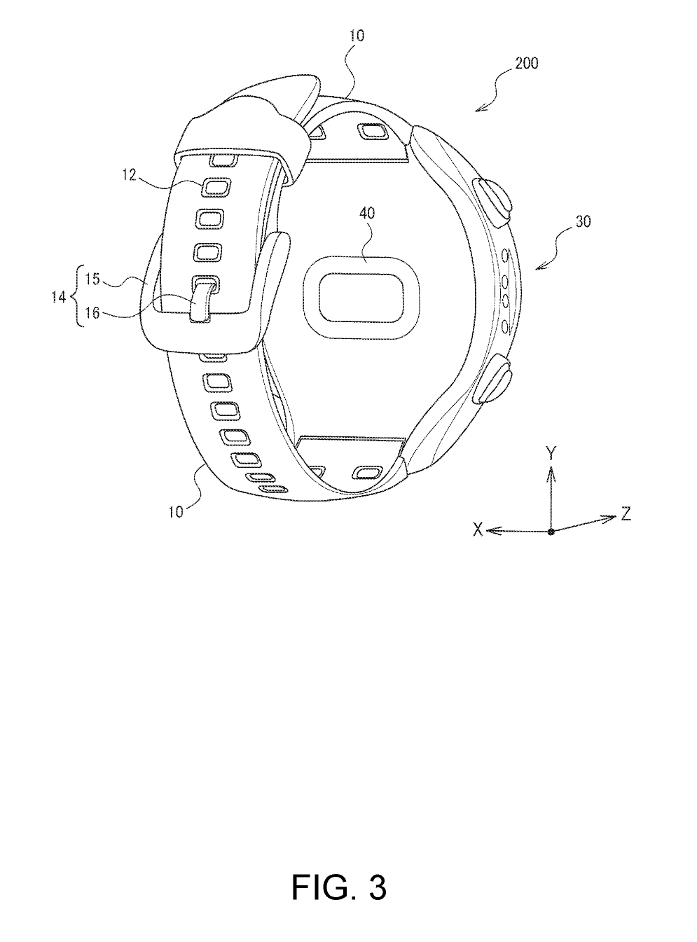

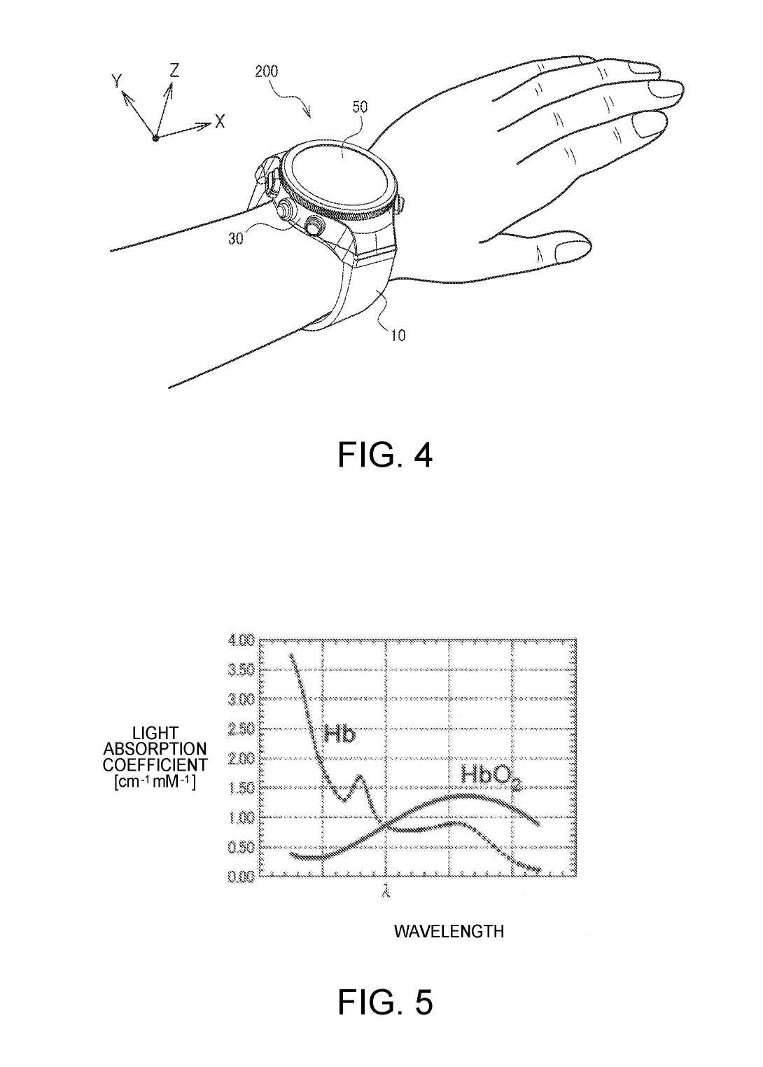

The measuring unit 110 measures biological information of the user doing activities in the closed space. It is desirable that a sensor which measures the biological information of the user is provided near or in contact with the user's body. Therefore, the measuring unit 110 may be implemented as a wearable device 200 mounted on the user, as described later with reference to FIGS. 3 and 4, for example. Alternatively, in a control device 300 provided with a processing unit 130, an interface which acquires information from a wearable device 200 may be used as a measuring unit 110. In this case, the measuring unit 110 is implemented as a receiving processing unit (communication unit) which receives information from the wearable device 200 via a network such as WAN (wide area network), LAN (local area network), or short-range wireless communication network.

The processing unit 130 carries out various kinds of processing on the basis of the biological information of the user measured by the measuring unit 110. The functions of the processing unit 130 can be implemented by hardware such as various processors (CPU or the like) and ASIC (gate array or the like), or by a program or the like.

FIG. 2 shows an example of the configuration of an environment chamber (space where the environmental state can be controlled, and in a narrow sense, hypoxic chamber) including the information processing system 100 according to the embodiment. As shown in FIG. 2, the environment chamber includes a closed space 60, a wearable device 200 worn by a user 70, and a control device 300 which controls the environmental state of the closed space 60.

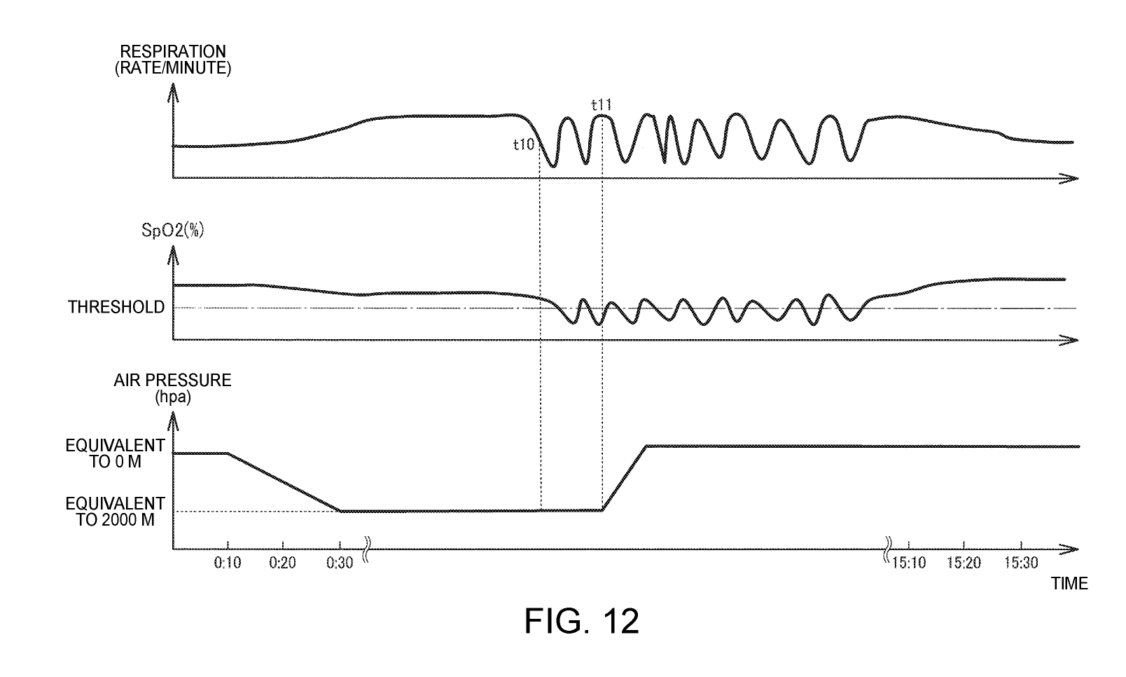

The closed space 60 is a space where the internal environment can be changed with respect to the external environment, as described above, and is highly airtight in the case of carrying out pressure control or concentration control. The user 70 can enter the inside of the closed space 60. A lighting unit 65, a training device 80 such as a treadmill, or a bed 90 which takes sleep into consideration, as described later with reference to FIGS. 11 and 12, or the like may be provided in the closed space 60.

The wearable device 200 is a device worn by the user 70 and includes a sensor unit 40 which detects at least biological information of the user.

FIG. 3 shows an example of the appearance of the wearable device 200. As shown in FIG. 3, the wearable device 200 includes a case part 30 and a band part 10 for fixing the case part 30 to a part (in a narrow sense, the wrist) of the user. The band part 10 is provided with a fitting hole 12 and a buckle 14. The buckle 14 is made up of a buckle frame 15 and an engaging part 16 (protruding rod) 16.

FIG. 3 is a perspective view showing the wearable device 200 in the state where the band part 10 is fixed using the fitting hole 12 and the engaging part 16, as viewed from the direction on the side of the band part 10 (on the side of the surface on the subject side in the wearing state, of the surfaces of the case part 30). In the wearable device 200 shown in FIG. 3, a plurality of fitting holes 12 is provided in the band part 10, and the engaging part 16 of the buckle 14 is inserted in one of the plurality of fitting holes 12, thus allowing the user to wear the wearable device 200. The plurality of fitting holes 12 is provided along the longitudinal direction of the band part 10, as shown in FIG. 3.

The sensor unit 40 is provided in the case part 30 of the wearable device 200. Here, since it is assumed that the sensor unit 40 is a photoelectric sensor including a light emitting unit and a light receiving unit, the sensor unit 40 is provided at a position exposed outside the case part 30 and particularly on the surface in contact with the subject side in the wearing state. That is, in the state where the wearable device 200 is worn, the sensor unit 40 is in tight contact with the living body. Therefore, by retraining external light from becoming incident on the light receiving unit or by reducing the distance between the sensor unit 40 and the living body, it is possible to reduce the optical path length and increase the detection signal intensity of the sensor unit 40.

FIG. 4 shows the wearable device 200 in the state of being worn by the user, as viewed from the side where a display unit 50 is provided. As shown in FIG. 3, the wearable device 200 according to the embodiment has the display unit 50 at a position corresponding to the dial of an ordinary wristwatch. In the state where the wearable device 200 is worn, the surface where the sensor unit 40 is provided, of the case part 30, is in tight contact with the subject, and the display unit 50 provided on the surface opposite to the sensor unit 40, of the case part 30, is at a position which is easily visible to the user.

Since it is assumed that various kinds of information are presented to the user 70 as an environment chamber user, FIG. 4 shows an example in which the wearable device 200 has the display unit 50. However, the notification to the user from the wearable device 200 is not limited to the display on the display unit 50, and light, vibration, sound and the like may be generated.

The control device 300 acquires information (sensor information, biological information) from the wearable device 200 and controls the environmental state of the closed space 60. The processing unit 130 is provided in the control device 300 and is implemented by a processor or the like included in the control device 300, for example. Also, the control device 300 includes a hardware configuration used for actual control of the environmental state, in addition to the processing unit 130.

For example, in the case of changing the pressure, the control device 300 includes a pump or the like capable of sucking and discharging gases. The pump or the like operates on the basis of a control signal from the processing unit 130. The control of the environmental state in the embodiment may be the control of air pressure or partial pressure of oxygen, that is, the control of pressure. Partial pressure of oxygen is decided by the product of the air pressure and the oxygen ratio in the gas. Therefore, in the case of achieving an environment where oxygen concentration is low (where partial pressure of oxygen is low), this can be done by lowering the air pressure itself or by lowering the air pressure and thus lowering the partial pressure of oxygen.

However, the low-oxygen state can be realized by concentration control, that is, by lowering the oxygen ratio in the gas, instead of the pressure control. When lowering the oxygen ratio in the gas, a gas (typically nitrogen) that is harmless to the human body other than oxygen may be introduced in the closed space 60.

That is, the environment where oxygen concentration is low may be a “low-pressure low-oxygen” environment where the air pressure itself is lowered, or a “normal-pressure low-oxygen” environment where the air pressure is maintained at 1 atmosphere (or a value close to it). In the case of the “normal-pressure low-oxygen”, since the difference between the air pressure in the closed space 60 and the external air pressure is small, the member forming the closed space 60 may have a relatively low strength, which is advantageous in that the member can be realized easily. Hereinafter, an example in which a “low-pressure low-oxygen” environment is realized by controlling air pressure will be described. However, it is possible to consider that the pressure control in the specification can be replaced by concentration control.

Meanwhile, in the case of changing the temperature or humidity in the closed space 60, the control device 300 may have equipment similar to air-conditioning equipment such as an air conditioner, dehumidifier, or humidifier, which is widely known. Also, in the case of changing the illuminance, the control device 300 may output a signal to control the lighting unit 65, for example.

Also, the information processing system 100 according to the embodiment may measure the environmental state of the closed space 60. The control of the environmental state by the control device 300 may be carried out by open-loop control. In such a case, whether a desired environmental state is actually achieved or not is unknown. Since the environmental state in the embodiment directly relates to health risks of the user, there is a great need to control the environmental state accurately. Therefore, the information processing system 100 according to the embodiment may perform closed-loop control in which the result of measuring the environmental state of the closed space 60 is acquired and where the environmental state is controlled on the basis of the result of the measuring.

Next, an example of a technique for detecting biological information and an example of the configuration of the sensor unit 40 will be described. The sensor unit 40 is a sensor for acquiring at least arterial blood oxygen saturation information and can be implemented by a photoelectric sensor, for example. The sensor unit 40 includes a light emitting unit which casts light with at least two wavelengths different from each other, and a light receiving unit which receives transmitted light which is the light from the light emitting unit transmitted through the subject, or reflected light which is the light from the light emitting unit reflected by the subject. As an example, the sensor unit 40 includes a first light emitting unit which casts light with a first wavelength, a second light emitting unit which casts light with a second wavelength, a first light receiving unit which receives transmitted light or reflected light from a living body, of the light from the first light emitting unit, and a second light receiving unit which receives transmitted light or reflected light from a living body, of the light from the second light emitting unit. The first wavelength is a wavelength corresponding to infrared rays and the second wavelength is a wavelength corresponding to red light, for example. However, the configuration of the sensor unit 40 is not limited to this and can be implemented with various modifications. For example, instead of providing two light receiving unit, a single light receiving unit may be used in a time-divisional manner.

FIG. 5 shows the light absorption spectrum of reduced hemoglobin Hb and the light absorption spectrum of oxidized hemoglobin HbO2. As shown in FIG. 5, the oxidized hemoglobin HbO2 and the reduced hemoglobin Hb have different light absorption spectra from each other. When light with a relatively long wavelength λ1 (>λ) is cast, the oxidized hemoglobin HbO2 has a greater light absorption coefficient for this light and therefore the intensity (output value V1 from the light receiving unit) of the transmitted light or reflected light from the living body, of this light, is an indicator value indicating the amount of oxidized hemoglobin in the blood vessel. Similarly, when light with a relatively short wavelength λ2 (<λ) is cast, the reduced hemoglobin Hb has a greater light absorption coefficient for this light and therefore the intensity (output value V2 from the light receiving unit) of the transmitted light or reflected light from the living body, of this light, is an indicator value indicating the amount of reduced hemoglobin in the blood vessel. Therefore, V1/(V1+V2) is an indicator value indicating the ratio of oxidized hemoglobin, that is, a value correlated with the blood oxygen saturation SpO2.

By utilizing the feature that the oxidized hemoglobin HbO2 and the reduced hemoglobin Hb have different light absorption spectra from each other, it is possible to find arterial blood oxygen saturation information from the transmitted light and reflected light of light with two different wavelengths. While a simple technique is described above, various modifications of the technique of finding SpO2 or information similar to SpO2 using infrared light and red light are known and these modifications can be broadly applied in the embodiment. Also, the wavelengths that are used are not limited to infrared light and red light, as long as the degrees of light absorption of the oxidized hemoglobin HbO2 and the reduced hemoglobin Hb for one wavelength are clearly different from the degrees of light absorption for the other wavelength. For example, modifications such as changing the light with one wavelength to green light can be made.

A traditional, broadly known portable pulse oximeter measures the arterial blood oxygen saturation (SpO2) in the state of being mounted on a fingertip. As a form of its use in an environment chamber, for example, the user wears the pulse oximeter on a fingertip and measures SpO2 to check his/her own state periodically or when the user perceives an abnormality in his/her physical condition. When SpO2 is lowered, the user takes measures such as taking a rest or suspending the training.

However, the traditional pulse oximeter is not assumed to be worn constantly. Specifically, in the SpO2 measurement in the traditional technique, the user needs to execute the procedures of temporarily stopping his/her activity, taking out the pulse oximeter from its storage site, mounting the pulse oximeter on a fingertip, and resting until the measurement is completed. In other words, the user cannot learn his/her SpO2 state without actively carrying out SpO2 measurement.

Such a technique has two problems. First, measuring SpO2 interrupts the user's activity (exercise). As described above, the broadly known pulse oximeter is a fingertip-mounted type and is not assumed to remain mounted during activities. Therefore, the user must temporarily stop activities such as training on a treadmill and measure SpO2. Also, during the measurement, it is difficult to grab something with the finger used for the measurement. In the case where the mounted part (measuring unit, sensor unit) on the fingertip and the main body part (processing unit, display unit) of the pulse oximeter are separate parts, the cable connecting the mounted part with the main body part becomes an obstacle, interrupting the user's activity.

Second, it is difficult to prevent symptoms of altitude sickness because there is a time lag from when the user falls in a hypoxic state to when symptoms of altitude sickness actually appear. For example, it is said that it takes a few hours to become aware of even headache, which is a relatively mild symptom, after falling in a hypoxic state. In the case of serious conditions such as cerebral edema and pulmonary edema, it takes a few days until the user develop these conditions after falling in a hypoxic state. That is, it is too late if the user measures SpO2 after becoming aware of his/her poor physical condition, and it is not useful for the prevention of altitude sickness. However, if the user tries to measure SpO2 very frequently, the user's activity such as training is interrupted each time, which is not preferable.

In this respect, if the wearable device 200 is used to measure biological information, SpO2 can be measured very frequently (in a narrow sense, constantly) without interrupting the user's activity and therefore this can cope with the above two problems. Specifically, by controlling the environmental state of the closed space 60 on the basis of the constantly measured SpO2, it is possible to contribute to the prevention of symptoms of altitude sickness. The wearable device 200 is a wrist-wearable device and may be mounted at a site such as an ankle or upper arm.

The sensor unit 40 may measure pulse wave information such as pulse rate. Since pulse waves appear as a change in the volume of blood, the pulse wave sensor measures pulse waves by catching a change in the amount of blood at the site to be measured. Considering that the amount of blood flow is correlated with the amount of hemoglobin in the blood, when light is cast on a blood vessel, as the amount of blood flow becomes greater and hence the amount of hemoglobin becomes greater, the amount of light absorbed becomes greater and the intensity of transmitted light or reflected light becomes lower. Conversely, as the amount of blood flow becomes smaller and hence the amount of hemoglobin becomes smaller, the amount of light absorbed becomes smaller and the intensity of transmitted light or reflected light becomes higher. That is, it is possible to detect pulse wave information on the basis of a change with time of the detection signal in the photoelectric sensor.

The light cast by the light emitting unit of the pulse wave sensor may preferably be of a wavelength that can be easily absorbed by hemoglobin, and green light is typically used. Thus, the wearable device 200 in the embodiment may have a sensor for detecting arterial blood oxygen saturation information and a pulse wave sensor separately. In this case, the wearable device 200 includes first and second light emitting units which cast light with a first wavelength (for example, infrared light) and a second wavelength (red light) for arterial blood oxygen saturation information, and a third light emitting unit which casts light with a third wavelength (green light) for pulse wave information. As for the light receiving unit, first to third light receiving units may be provided corresponding to the first to third light emitting units. Also, one or two light receiving units may be provided and used in a time-divisional manner.

Alternatively, pulse wave information may be detected using the sensor for detecting arterial blood oxygen saturation information. Specifically, one of the first light emitting unit and the second light emitting unit of the sensor is used as a light emitting unit for detecting pulse wave information as well. In this case, since the light has two wavelengths, pulse wave information is detected using red light, and arterial blood oxygen saturation information is detected using infrared light and red light. Alternatively, pulse wave information is detected using green light, and arterial blood oxygen saturation information is detected using infrared light and green light. Moreover, various modifications can be made to the wavelength of the light.

Also, the pulse wave information is not limited to pulse rate and may be pulse interval (RR interval), variation in pulse interval, or other information indicating pulse waves. It is also possible to acquire information about respiration of the user from a variation in the pulse wave information. Therefore, the wearable device 200 may acquire respiration information as biological information, on the basis of the information from the pulse wave sensor. However, respiration information can also be measured using a broadly known breath analysis sensor.

The wearable device 200 may also include a temperature sensor and may measure the body temperature of the user on the basis of an output from the temperature sensor. However, a technique of estimating temperature using an infrared sensor or the like is known as well, and in such a case, the temperature sensor may be provided in a place other than the wearable device 200.

The wearable device 200 may also include a motion sensor (body movement sensor) which detects movements of the user, such as an acceleration sensor or gyro sensor. The measuring unit 110 may find the intensity of exercise of the user, type of exercise, number of steps taken in walking or running, and pitch, on the basis of the motion sensor. Alternatively, the measuring unit 110 may find the amount of activity of the user in terms of calories burned, Mets or the like. Moreover, the wearable device 200 may include an environment sensor capable of measuring environment information of the peripheries, for example, a temperature sensor, humidity sensor, air pressure sensor, light amount sensor, and audio sensor (microphone or like).

### 3. Pressure Change Control

Next, a specific example of control in the embodiment will be described. Here, an example in which the biological information is arterial blood oxygen saturation information (particularly SpO2) and in which the environment state controlled on the basis of the biological information is the pressure (air pressure) in the closed space 60 will be described.

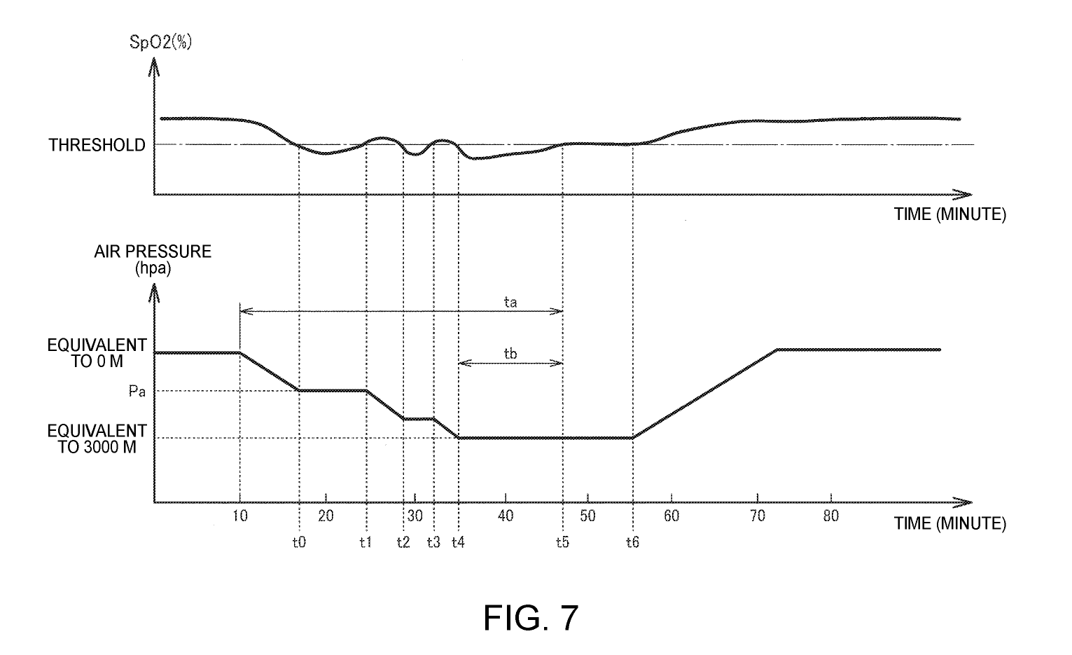

FIG. 6 shows an example of the profile for pressure control in the related-art technique. In FIG. 6, the horizontal axis represents time (minute) and the vertical axis represents air pressure. In FIG. 6, at the start point, the air pressure is equivalent to that at 0 meters above sea level. During the 20 minutes from the point of 10 minutes to the point of 30 minutes, the air pressure is reduced to be equivalent to that at 3000 meters above sea level. During the 20 minutes from the point of 30 minutes to the point of 50 minutes, the air pressure equivalent to that at 3000 meters above sea level is maintained. Then, during the 20 minutes from the point of 50 minutes to the point of 70 minutes, the air pressure is returned (increased) from the air pressure equivalent to that at 3000 meters above sea level to the air pressure equivalent to that at 0 meters above sea level. While various forms are known as the training in a hypoxic chamber that are currently in practice, in short-term training, one session of training is often completed in approximately several ten minutes to an hour. For example, carrying out the training shown in FIG. 6 once every few days over several weeks enhances cardiopulmonary function. Here, while the air pressure at the start point is expressed as the “air pressure equivalent to that at 0 meters above sea level”, the air pressure at the start point is not limited to this. The air pressure at the start point may be the atmospheric pressure in the place or a predetermined air pressure set by the operator or user. Also, while the vertical axis in FIG. 7 represents air pressure (hpa) in order to facilitate understanding, environmental control of the closed space may be carried out by employing oxygen concentration (hpa, %) on the vertical axis, instead of air pressure.

However, in FIG. 6, the air pressure at each timing is set, and in the control of air pressure, the air pressure value at each timing is a numerical value prescribed by the profile of FIG. 6. That is, as described above, since the state of the user 70 is not related to the air pressure control, even with the control according to the profile of FIG. 6, some users can fall into a hypoxic state and develop symptoms of altitude sickness or fall into a state where the development of these symptoms is highly likely.

The occurrence of a health risk to the user 70 is due to the fact that the environmental state of the closed space 60 where the user 70 carries out activities changes too quickly for the response (acclimatization) of the user 70 to catch up. When there can be a health risk, the biological information of the user 70 is considered to change in a certain way corresponding to the health risk in question. Therefore, in the embodiment, the processing unit 130 decides whether to change the environmental state on the basis of the biological information or not.

Specifically, if the value of the biological information satisfies a predetermined control execution condition (on condition that the value satisfies the control execution condition), the processing unit 130 carries out control to change the environmental state of the closed space 60. As the arterial blood oxygen saturation information (SpO2) becomes higher, there are fewer problems with the state of the user, whereas if the arterial blood oxygen saturation information (SpO2) becomes lower, it is suspected that the user has more problems or is more likely to develop symptoms of altitude sickness. Therefore, in the case of using arterial blood oxygen saturation information, the processing unit 130 carries out control to change the environmental state of the closed space 60 if the value of the arterial blood oxygen saturation information is equal to or above a predetermined threshold (on condition that the value is equal to or above the predetermined threshold). The control to change the environmental state in this case is the control to change the pressure in the closed space 60. The control execution condition in this case is a pressure change allowing condition. In the description below, the control execution condition is assumed to be the pressure change allowing condition. However, in the case of controlling concentration as the environmental state, the control execution condition is a concentration change allowing condition. In such a case, the processing unit 130 carries out control to change the concentration in the closed space 60 if the value of the biological information satisfies a predetermined control execution condition (concentration change allowing condition).

FIG. 7 shows a specific profile for pressure control in the case where control is carried out by the technique of the embodiment. FIG. 7 shows change with time in the value of SpO2, which is biological information, and change with time in the air pressure in the closed space 60 controlled on the basis of SpO2. In FIG. 7, the horizontal axis represents time and the vertical axis represents the value of SpO2(%) and air pressure. The control in the embodiment may also be implemented, for example, by correcting the profile as shown in FIG. 6 through a determination based on biological information.

In the example of FIG. 7, as in the example of FIG. 6, pressure reduction is started at the point of 10 minutes, with a target pressure equivalent to that at 3000 meters above sea level from the air pressure equivalent to that at 0 meters above sea level. With this pressure reduction, SpO2 of the user drops, as shown in FIG. 7. Then, when the SpO2 value drops below a threshold (at a timing t0), the pressure change allowing condition is no longer satisfied. That is, the user cannot cope with the change in pressure and consequently it is determined that the SpO2 is below the threshold. If pressure reduction is continued further in this state, the user falls into a hypoxic state and has a higher risk of developing symptoms of altitude sickness.

Therefore, if the value of the biological information does not satisfy the control execution condition, the processing unit 130 carries out control to maintain the pressure or concentration in the closed space 60. Here, if the value of the biological information does not satisfy the pressure change allowing condition, the processing unit 130 carries out control to maintain the pressure in the closed space 60. In the example of FIG. 7, after the timing t0, when SpO2 drops below the threshold, the pressure at the timing t0 is maintained and pressure reduction is not carried out. In this way, the environmental state can be restrained from being changed too quickly for the user to cope with. That is, the user can be allowed to safely carry out his/her activities in the environment chamber (in the closed space 60).

As the control to maintain the pressure is carried out, it is considered that the user gradually acclimatizes to this pressure and that the SpO2 value gradually increases. Subsequently, when SpO2 increases to or above the threshold (timing t1), the pressure change allowing condition is satisfied. Therefore, pressure reduction is started at the timing t1, again with a target air pressure equivalent to that at 3000 meters above sea level.

SpO2 is considered to drop as pressure reduction is resumed. In some cases, SpO2 drops below the threshold again and pressure change is restrained. In the example of FIG. 7, since SpO2 drops below the threshold at a timing t2, the pressure at the timing t2 is maintained and the recovery of SpO2 is waited for. Then, at a timing t3, SpO2 rises to or above the threshold and therefore pressure reduction is started again. The pressure reduction to a target air pressure equivalent to that at 3000 meters above sea level is completed at a timing t4.

Also, even if the biological information satisfies the pressure change allowing condition, the pressure need not be changed beyond a target value. Considering the safety of the user, an excessive pressure change should be avoided. That is, not only whether the biological information satisfies the pressure change allowing condition or not, but also whether the current pressure has reached a predetermined set value (target value) or not, is a condition for changing the pressure. In other words, the processing unit 130 carries out control to change the pressure or concentration in the closed space if the value of the biological information satisfies the control execution condition and the pressure or concentration in the closed space 60 has not reached a predetermined set value. Here, the processing unit 130 carries out control to change the pressure in the closed space if the value of the biological information satisfies the pressure change allowing condition and the pressure in the closed space has not reached a predetermined set value.

The set value in this case corresponds to the air pressure equivalent to that at 3000 meters above sea level, which is the target value in the pressure reduction in the example of FIG. 7. However, the set value is not limited to this. For example, a target value in pressure increase may be used as a set value. Alternatively, pressure reduction may be divided into a plurality of phases and executed phase by phase, and a target value in each phase may be used as a set value. Alternatively, to make more precise adjustment, a target pressure may be set for each timing and this may be used as a set value.

Therefore, for example, at the timing t5, though SpO2 is equal to or above the threshold and therefore satisfies the pressure change allowing condition, the control to change the pressure is not carried out. This is because the timing t5 is included in the stage where the air pressure equivalent to that at 3000 meters above sea level is maintained for 20 minutes and the current air pressure has reached the set value, which is, in this case, the air pressure equivalent to that at 3000 meters above sea level.

While the pressure reaches the set value at the timing t4, SpO2 is considered to temporarily drop to below the threshold due to the influence of the pressure reduction from the timing t3 to t4. Therefore, in the example of FIG. 7, SpO2 is below the threshold during the period from the timing t4 to t5.

As shown in FIG. 7, from the timing t6 after the lapse of 20 minutes from the timing t4, pressure increase control to return the air pressure to the air pressure equivalent to 0 meters above sea level in 20 minutes is carried out, as in the example shown with reference to the period from the point of 50 minutes to the point of 70 minutes in FIG. 6. After the timing t6, the oxygen concentration in the closed space 60 rises with time. That is, SpO2 is considered to rise with pressure change.

Thus, it is considered unlikely that the biological information (arterial blood oxygen saturation information) no longer satisfies the pressure change allowing condition as shown in FIG. 7, and the control toward pressure increase ends in 20 minutes from the timing t6 as the start point. However, in the pressure increase, too, if a sudden pressure change occurs in a short time, this causes a heavy burden on the user. Therefore, here, pressure increase control is carried out over a period of 20 minutes. In other words, it can be said that the time taken for a second process of changing from a predetermined air pressure to the air pressure at the start point is shorter than the time taken for a first process of changing from the air pressure at the start point to the predetermined air pressure. It can also be said that the speed of change in the air pressure or oxygen concentration in the second process is higher than the speed of change in the air pressure or oxygen concentration in the first process. With such control, the air pressure can be controlled within a minimum necessary time.

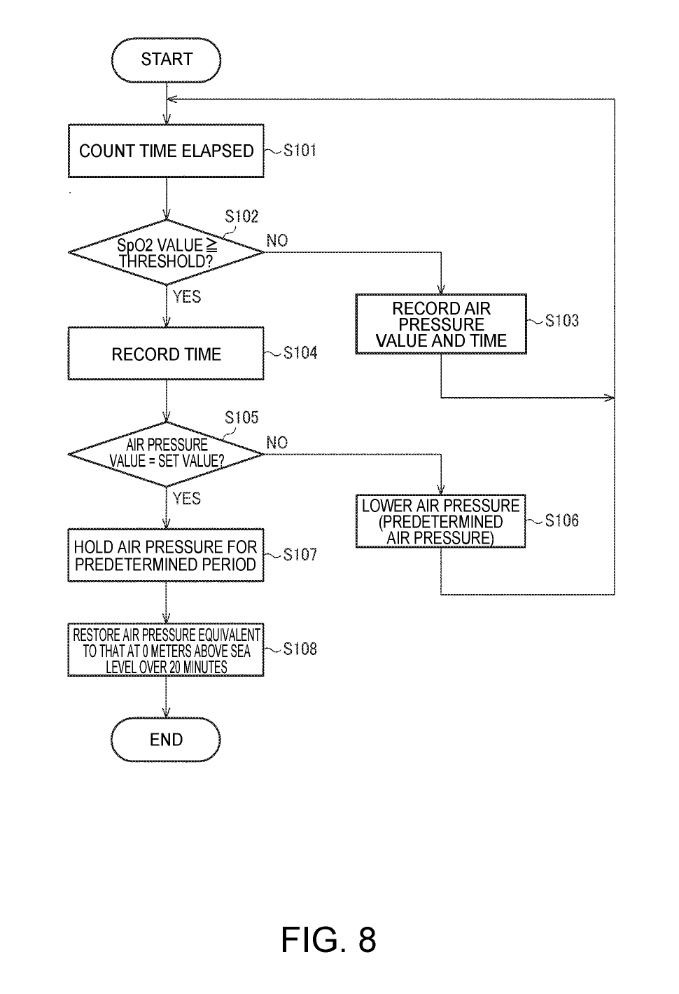

FIG. 8 is a flowchart for explaining the control in the embodiment described above with reference to FIG. 7. Each step of processing in FIG. 8 is executed by the processing unit 130. As this processing is started, first, the processing unit 130 counts the time elapsed (S101). The counting of the time elapsed may be carried out using a timer provided in the information processing system 100 itself, or may be carried out by acquiring time information from an external device via a network or the like.

Then, the processing unit 130 determines whether the biological information satisfies the pressure change allowing condition. Here, the processing unit 130 determines whether the SpO2 value is equal to or above a threshold (S102). If the result is No in S102, the control to change the pressure cannot be executed. Therefore, the air pressure value at the time and the time are stored (S103) and the processing returns to S101.

If the result is Yes in S102, the time is stored (S104) and the processing unit 130 determines whether the air pressure value has reached a set value (S105). In FIG. 8, the set value is a target value of pressure reduction and a numerical value expressing the air pressure equivalent to that at 3000 meters above sea level. If the result is No in S105, this is equivalent to the case where the pressure change allowing condition is satisfied and where the current air pressure has not reached the set value. Therefore, the processing unit 130 carries out control to lower the air pressure (S106). As described above, since a sudden pressure change increases health risk to the user, control that does not cause an excessive increase in the amount of pressure change in S106 may be carried out. For example, the value of reduction in pressure per unit time in S106 may be a predetermined value.

If the result is Yes in S105, this means that the pressure reduction to the target set value has been completed. Therefore, this air pressure is maintained for a predetermined period (S107). The predetermined period in S107 is 20 minutes in the example of FIG. 7. Moreover, pressure increase control to restore the air pressure in normal time, that is, the air pressure equivalent to that at 0 meters above sea level, is carried out over a period of 20 minutes (S108). Then, a series of pressure controls ends.

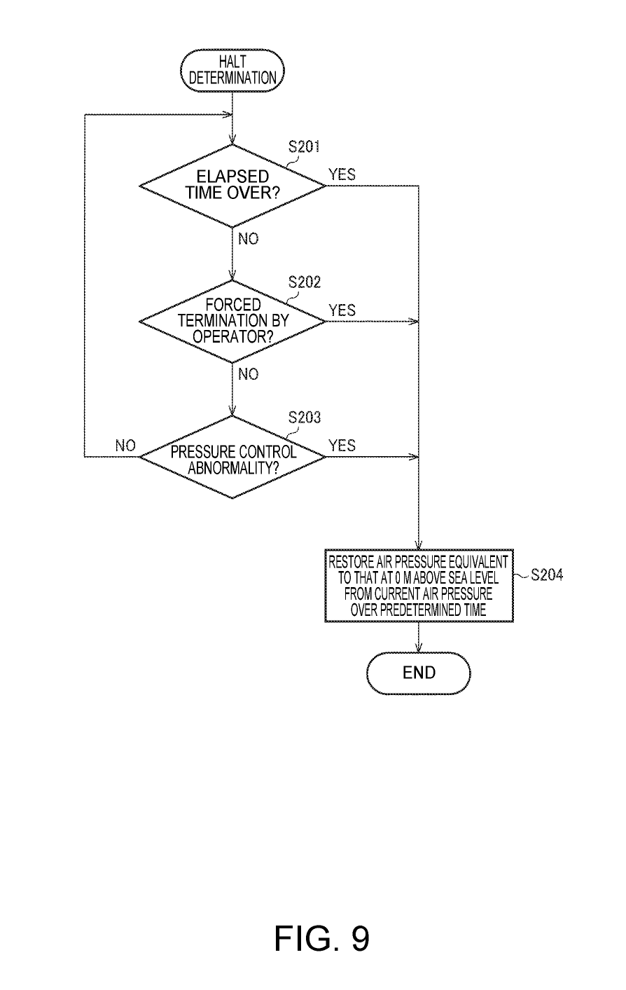

Apart from the above processing, the processing unit 130 monitors whether an abnormality that requires a forced halt on pressure control is generated or not. FIG. 9 is a flowchart for explaining forced halt determination. In the forced halt determination, the processing unit 130 carries out a time-out determination on whether the time elapsed is longer than a predetermined time (S201), a determination on whether a forced termination is inputted by the operator (S202), and a determination on whether an abnormality is generated in the pressure control by the control device 300 (S203). If the result of the determination is Yes in at least one of S201 to S203, that is, if it is determined that at least one of time-out, force termination input, and pressure control abnormality is generated, further continuation of the training is dangerous and therefore the processing unit 130 forced-halts the series of pressure controls.

Specifically, the air pressure equivalent to that at 0 meters above sea level is restored from the current air pressure over a predetermined period of time (S204). In this case, while it is desired that the normal state is restored as quickly as possible to secure the safety of the user, a sudden pressure change applies a load on the user and therefore is not preferable. Thus, various settings of the predetermined time in S204 are possible according to the circumstances. For example, pressure change control from the current air pressure may be executed at the rate of change in the case of changing the air pressure from the air pressure equivalent to that at 3000 meters above sea level to the air pressure equivalent to that at 0 meters above sea level over 20 minutes, as in S108.

### 4. Acclimatization Indicator

The environment chamber (hypoxic chamber) including the information processing system 100 according to the embodiment is used to enhance cardiopulmonary function, or the like. Therefore, there is a great demand by the user for the knowledge of the extent of effect to which the training using the environment chamber has proved. However, with the related-art technique, no clear training effects are presented, and for example, the user has to determine whether his/her time has improved by actually running a long distance. Therefore, the confirmation of effects takes effort, and even if the user's time has improved, it is difficult to determine whether the improvement is the effect of the training in the environment chamber or the effect of other trainings.

Thus, in the embodiment, information that can present to the user the effect of an activity using the environment chamber in a way that is easy to understand is found. Specifically, the processing unit 130 finds an acclimatization indicator of the user on the basis of the biological information of the user. Hereinafter, an example of a pressure change acclimatization indicator, which is an acclimatization indicator of the user to a change in pressure, will be described.

As described above, the biological information is information indicating a change in the state of the user with respect to an environmental change. That is, whether the user can quickly cope with an environmental change or takes time to cope with the environment change can be determined on the basis of the biological information that is actually measured. Particularly, since the biological information is used for the determination on whether to change the environmental state or not, specifically, for the determination on the pressure change allowing condition, there is an advantage that the same biological information can also be used for the calculation of a pressure change acclimatization indicator.

Various techniques are conceivable with respect to what pressure change acclimatization indicator is to be found on the basis of the biological information. For example, when the environment is gradually changed, what extent of environmental change is tolerable for the biological information to be maintained within a normal range can be one indicator. If the user can tolerate a greater change, it can be determined that the acclimatization has made further progress and that the effect of the training has been achieved. Alternatively, when the user is in a high-load environment and the timing when the biological information falls into a normal range, can also be used as an indicator. Also, when the environment is gradually changed, a pressure change acclimatization indicator can be found and the effect of the training can be determined on the basis of whether the amount of change in the biological information is smaller than a predetermined value, or whether the amount of change in the biological information is within a predetermined range or not. If the user is under a high load but has no abnormality in the biological information, it means that the adjustment to the environmental change has been completed. Therefore, if the user can shift to such a state more quickly, it can be determined that the acclimatization has made further progress and that the effect of the training has been achieved.

As described above with reference to FIG. 7, in the case of the example of pressure change, the processing unit 130 may find, as the pressure change acclimatization indicator of the user, at least one of pressure information about the pressure in the case where the value of the biological information no longer satisfies the pressure change allowing condition, and time information about the timing when the value of the biological information satisfies the pressure change allowing condition after the pressure reaches a set value.

In FIG. 7, the pressure information about the pressure in the case where the value of the biological information no longer satisfies the pressure change allowing condition is a pressure pa or information based on the pressure pa. The pressure pa indicates the value of the pressure at the first time the biological information no longer satisfies the pressure change allowing condition (timing t1) after the environmental change (pressure reduction) is started. As the value of pa becomes smaller, it means that the biological information of the user can be maintained in the normal range even if the pressure drops, and that the acclimatization has made progress. Alternatively, a differential value between the pressure at the start of pressure reduction and pa may be used instead of the absolute value of the pressure. In such a case, as the difference becomes greater, it means that the user can tolerate a greater pressure change and that the acclimatization has made progress.

In short, the processing unit 130 finds, as the pressure information which is the pressure change acclimatization indicator, at least one of the value of the pressure in the case where the value of the biological information no longer satisfies the pressure change allowing condition, and the difference information indicating the change in the pressure until the value of the biological information no longer satisfies the pressure change allowing condition from before the start of the change in pressure. The value of the pressure is the above value pa. The difference information is information about the difference between the air pressure equivalent to that at 0 meters above sea level and pa.

In this way, as the value of the pressure becomes smaller or as the difference expressed by the difference information becomes greater, it is understood that the acclimatization of the user has made progress. Therefore, it is possible to present to the user the effect of carrying out activities using the environment chamber, in a way that is easy to understand, or the like.

Also, in FIG. 7, the timing when the value of the biological information satisfies the pressure change allowing condition after the pressure reaches the set value corresponds to the timing t5. That is, in the example of FIG. 7, it is considered that the user's adjustment to the high-load environmental state is completed at the timing t5. As described above, it is preferable that the adjustment to the high-load environment is completed as quickly as possible. Therefore, ta or tb shown in FIG. 7 may be used as an indicator value. The value ta expresses the time period from the start of pressure reduction to the timing t5, and tb expresses the time period from the timing (timing t4) when the pressure reaches the set value to the timing t5.

In short, the processing unit 130 finds, as the time information which is the pressure change acclimatization indicator, at least one of first time information ta expressing the time period from the timing when the pressure change is started to the timing when the value of the biological information satisfies the pressure change allowing condition after reaching the set value, and second time information tb expressing the time period from the timing when the pressure reaches the set value to the timing when the value of the biological information satisfies the pressure change allowing condition.

The first time information ta expresses the time taken to adjust to the overall pressure change from before the start of pressure reduction to the completion of the pressure reduction. The second time information tb expresses the time taken to adjust to the environment where the user is exposed to the low air pressure of the set value. Whichever time information is used, it is understood that the acclimatization of the user has made progress as this time becomes shorter. Therefore, it is possible to present to the user the effect of carrying out activities using the environment chamber, in a way that is easy to understand, or the like.

The pressure change acclimatization indicator may be an indicator indicating a value found from one session of training. For example, a standard value may be found in advance and the cardiopulmonary function of the user may be presented due to the value which is higher or lower than the standard value in a way that is easy to understand. Alternatively, data of many users may be held in advance and what position a target user is in, compared with other users, may be presented. Also, a target value or target range of biological information in a predetermined environment may be set according to the goal of the user, and the value of the biological information measured at the time of training may be compared with the target value or target range. Thus, not only the effect of the training but also the state of progress or degree of target achievement can be presented.

However, as described above, it is conceivable that training using the environment chamber is executed repeatedly to a certain extent. Therefore, information about change with time in the pressure change acclimatization indicator, that is, how the pressure change acclimatization indicator has changed as the result of repeated training by the same user, is very important as well.

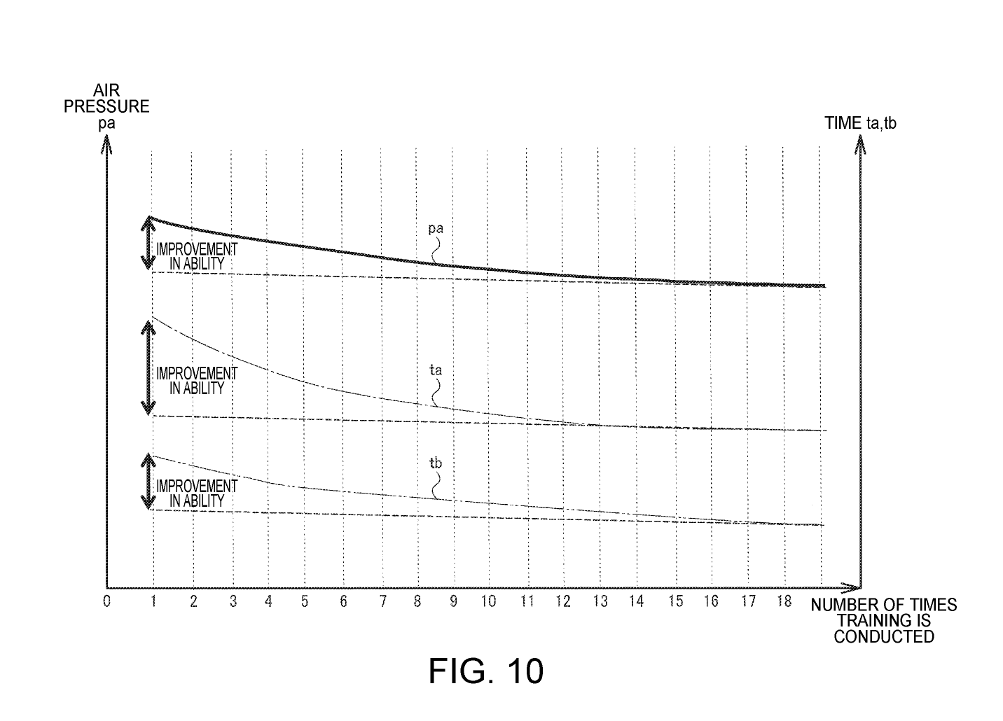

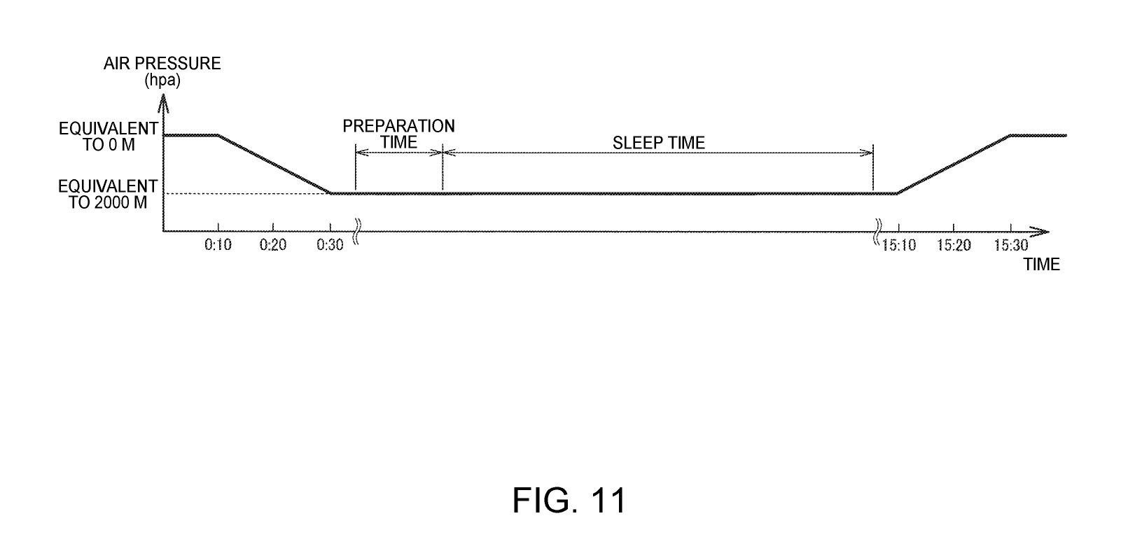

FIG. 10 shows an example of change in the pressure change acclimatization indicator due to continuous training. In FIG. 10, the horizontal axis represents the number of times the training is conducted, and the vertical axis represents the value of the pressure change acclimatization indicator. Here, the value pa of the pressure, the first time information ta, and the second time information tb are used as pressure change acclimatization indicators. In the example of FIG. 10, the values pa, ta, and tb decrease as the training is repeated. That is, by the presentation of the time-series change in the values pa, ta, and tb, the extent to which the acclimatization to the pressure change has made progress and the extent to which the effect of the training is achieved can be presented to the user in a way that is easy to understand. The processing unit 130 may perform display processing of FIG. 10 itself or may present information about the pressure change acclimatization indicators in different forms.

As shown in FIG. 10, the degree of improvement in ability corresponds to the amount of decrease in the values pa, ta, and tb. Therefore, the processing unit 130 may find the amount of change in at least one of the values pa, ta, and tb, or similar information, as the pressure change acclimatization indicator.

As described above, the pressure in the above description can be replaced with concentration (oxygen concentration). The processing unit 130 may find concentration information about the concentration in the case where the value of the biological information no longer satisfies the control execution condition (concentration change allowing condition), as the acclimatization indicator of the user. Specifically, the processing unit 130 may find, as the concentration information which is the acclimatization indicator, at least one of the value of the concentration in the case where the value of the biological information no longer satisfies the control execution condition, and difference information indicating a change in the concentration during the period until the value of the biological information no longer satisfies the control execution condition from before the start of the concentration change.

### 5. Modifications

Several modifications will be described below.

5.1 Example Using Biological Information Other than SpO2

A technique using arterial blood oxygen saturation information, particularly the SpO2 value, as biological information, is described above. However, the biological information used in the embodiment is not limited to arterial blood oxygen saturation information and may be other information. For example, respiration information of the user may be used along with SpO2 as the biological information. The respiration information may be acquired using a respiratory function testing apparatus such as a spirometer or may be found on the basis of pulse wave information. For example, the respiratory state can be estimated using an envelope of a pulse waveform or the like.

FIG. 11 shows a standard profile for pressure control in the case of carrying out hypoxic training involving sleep. In actual altitude training, the athlete stays at high altitude for several weeks and enhances his/her cardiopulmonary function while repeating daytime activities (exercise) and sleep. Therefore, in the case of using a hypoxic chamber, a technique of continuously staying in the hypoxic chamber for a longer time than in the example of FIG. 6 and having a sleep during training is conceivable. For example, as shown in FIG. 11, the air pressure is reduced from the air pressure equivalent to that at 0 meters above sea level to a set value (here, the air pressure equivalent to that at 2000 meters above sea level) over a period of 20 minutes, and the state where the air pressure is the set value is maintained for a relatively long time of approximately 15 hours. Then, the athlete has a sleep during the period when the air pressure is at the set value. After the athlete wakes up, the air pressure equivalent to that at 0 meters above sea level is restored over a period of 20 minutes.

However, sleep disorders are included as specific symptoms of altitude sickness. Therefore, as can be seen in actual altitude training, the athlete may be unable to sleep well in the environment with low oxygen and consequently his/her physical condition may become compromised.

Thus, if the activity in the closed space 60 such as training involves sleep, the processing unit 130 may determine whether to return the pressure to the reference value or not, on the basis of the biological information corresponding to the sleeping state of the user. The determination on whether the user has a proper sleep or not is important for achieving efficient training, and the determination about sleep enables safer execution of training. Also, if it is determined that the user in a dangerous state, it is possible to secure the safety of the user by returning the pressure to the reference value (for example, the air pressure equivalent to that at 0 meters above sea level). That is, by measuring the hours of sleep, depth of sleep, quality of sleep, respiratory state and the like as well as the SpO2 value on the basis of the biological information, it is possible to grasp the state of the user more accurately.

FIG. 12 shows a specific profile for pressure control in the case where control is performed using the technique according to this modification. FIG. 12 shows change with time in respiration information and SpO2 as biological information, and change with time in the air pressure in the closed space 60 controlled on the basis of biological information. In FIG. 12, the horizontal axis represents time, and the vertical axis represents respiratory rate per minute, SpO2 value (%), and air pressure.

First, the processing unit 130 performs control to reduce pressure until the air pressure reaches a set value, starting at the point of 10 minutes, as shown in FIG. 12. In this case, as described above with reference to FIG. 7, SpO2 being equal to or above a threshold is the condition for pressure change. In the example of FIG. 12, since SpO2 does not drop below the threshold before the air pressure equivalent to that at 2000 meters above sea level is reached, the pressure continuously decreases.

After the pressure reaches the set value, the user has a preparation time and then begins to sleep. Considering the effect of the training, it is desirable that the user begins to sleep after the pressure reaches the set value. Therefore, the processing unit 130 determines whether to return the pressure to the reference value or not, on the basis of the biological information after the pressure in the closed space reaches the set value.

Specifically, the processing unit 130 performs control to return the pressure to the reference value if it is determined that the number of times the value of the biological information satisfies a predetermined interruption condition is a predetermined number of times or above. In an example shown in FIG. 12, the biological information means the respiration information and SpO2. The interruption condition corresponds to the respiration and SpO2 in the state of hypopnea (in a narrow sense, apnea).

In the case of hypopnea or apnea, the respiratory rate per unit time decreases. Also, as the respiratory rate drops, oxygen in the blood decreases and the SpO2 value drops as well. Therefore, the interruption condition in this case may be, for example, that both the respiratory rate and the SpO2 value have dropped. More specifically, if the respiratory rate is equal to or below a threshold and SpO2 is equal to or below a threshold, it may be determined that the interruption condition is satisfied. The threshold for SpO2 in this case may be the same as or different from the threshold used in the embodiment of FIG. 7, that is, the same as the threshold in determining the pressure change allowing condition. In other words, the interruption condition may be the same as or different from the pressure change allowing condition.

As shown in FIG. 12, the timing when the respiratory rate drops and the timing when SpO2 consequently drops are different. Therefore, it is desirable that the interruption condition is satisfied even if the drop in the respiratory rate and the drop in SpO2 do not occur at the same timing. For example, it may be determined that the interruption condition is satisfied if one of the respiratory rate and SpO2 drops within a predetermined time after the other value drops.

Also, it is known that even a healthy user (user without sleep disorders) may experience hypopnea or apnea a few times an hour during sleep. That is, even if the respiratory rate and SpO2 drop, if the number of times it occurs is small, this cannot be considered as a serious situation where training must be interrupted. Therefore, in the embodiment, the number of times the interruption condition is satisfied is counted, and if the number of times exceeds a predetermined value, the training is forced-halted. More specifically, if the interruption condition is satisfied a predetermined number of times or more within a predetermined period of time, or if the number of times the interruption condition is satisfied is cumulatively counted from the beginning of sleep (or the beginning of training) and the cumulative number of times becomes equal to or above a predetermined number of times, the processing unit 130 performs control to forced-halt the training. In the example of FIG. 12, since it is detected that a drop in the respiratory rate and the SpO2 value occurs a predetermined number of times during the period from t10 to t11, the processing unit 130 performs control to forced-halt the training and return the air pressure from the current value to the air pressure equivalent to that at 0 meters above sea level over a predetermined time such as 20 minutes.

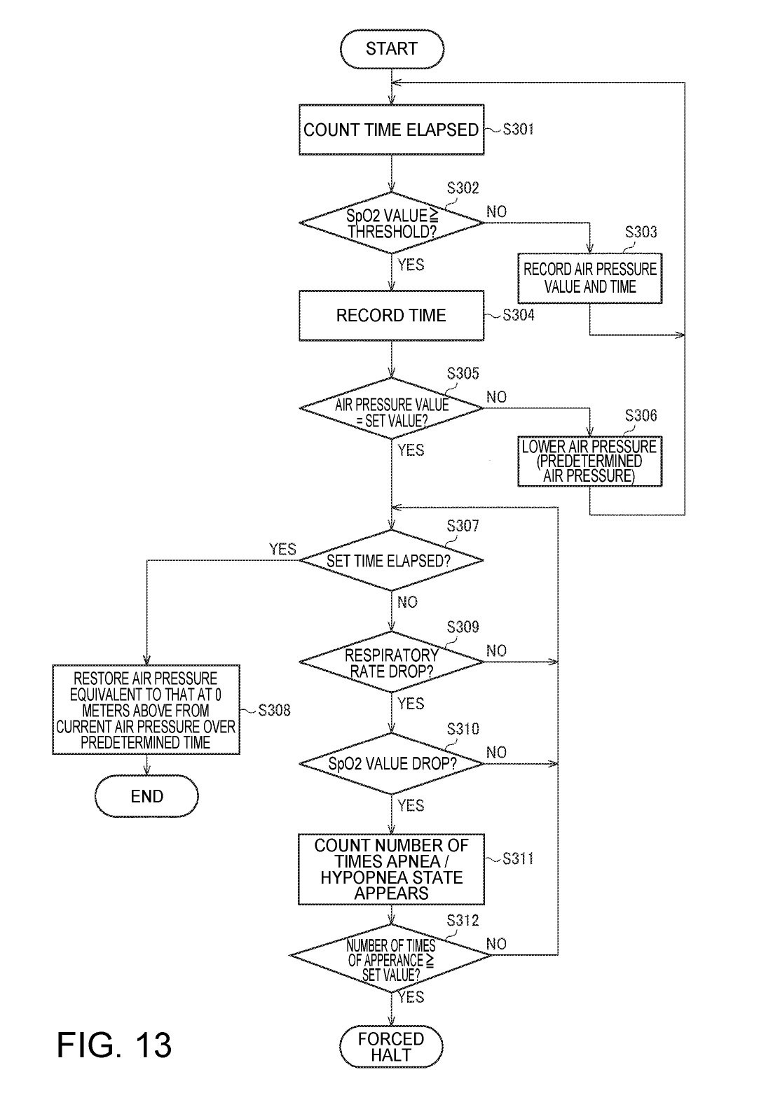

FIG. 13 is a flowchart for explaining the control in the embodiment described above with reference to FIG. 12. Each step of processing in FIG. 13 is executed by the processing unit 13. S301 to S306 in FIG. 13 are the steps of pressure reduction control similar to S101 to S106 in FIG. 8 and therefore will not be described further in detail.

If the result is Yes in S305, it means that the pressure reduction to the target set value is completed. Therefore, whether the set time has elapsed or not (whether the air pressure of the set value has been maintained for a predetermined period or not) is determined (S307). If the result is Yes in S307, the air pressure is returned to the air pressure equivalent to that at 0 meters above sea level over a predetermined time and then the training is ended normally (S308), similarly to S108 in FIG. 8. If the result is No in S307, the planned profile is not finished, and therefore whether the training can be continued without problems or not is determined.

Specifically, the determination on whether the respiratory rate has dropped or not (S309) and the determination on whether the SpO2 value has dropped or not (S310) are carried out, as described above. If the result is No in at least one of S309 and S310, there is no problem with continuing the training and therefore the processing returns to S307. If the result is Yes in both S309 and S310, the number of times the apnea/hypopnea state has emerged is counted (S311) and whether the number of times is equal to or above a predetermined threshold (S312).

If the result is No in S312, the apnea/hypopnea state may have occurred but not to an extent that requires immediate execution of a forced halt. Therefore, back to S307, the processing continues. If the result is Yes in S312, it is determined that the user has not had a proper sleep and therefore a forced halt is executed. The forced halt is implemented by the control to return to the air pressure equivalent to that at 0 meters above sea level from the current air pressure over a predetermined time, similarly to S204 in FIG. 9. In the case of FIG. 13, too, the processing unit 130 monitors time-out, forced termination input, and pressure control abnormality, similarly to S201 to S203 in FIG. 9, and if it is determined that at least one of these has occurred, the processing unit 130 forced-halts the series of pressure controls. That is, in the example of FIG. 13, it can also be understood that the forced halt determination based on the sleep state of the user is executed in addition to the forced halt determination shown in FIG. 9.

While an example using respiration information as biological information other than arterial blood oxygen saturation information is described above, other biological information can also be used. For example, pulse wave information such as pulse rate or pulse interval may be used, or information about body temperature or perspiration may be used.

### 5.2 Pressure Increase Control

The technique of determining the pressure change allowing condition in the pressure control to lower the pressure in the closed space 60 (for pressure reduction) is described above. However, the technique in the embodiment is not limited to this example. The pressure change allowing condition may be determined at the time of pressure increase as well, and the pressure may be increased on condition that the pressure change allowing condition is satisfied. In the case of pressure increase, too, a change in the pressure causes a load on the user. However, by using this technique, it is possible to increase the pressure safely.

For example, as in the pressure increase for 20 minutes from the timing t6 in FIG. 7, the pressure change allowing condition may be determined during the period of restoration to the normal state in training in a hypoxic chamber. However, when the pressure is increased, it is considered that the oxygen concentration becomes relatively high and that the SpO2 value rises. The upper limit of the SpO2 value is 100% and therefore does not have an excessively large value even if the oxygen concentration becomes too high. Therefore, in the case of setting a pressure change allowing condition at the time of pressure increase, it is desirable to use different biological information from SpO2.

The pressure increase in this case is not limited to the pressure increase in a hypoxic chamber but may be the pressure increase in an environment chamber which achieves a hyperoxic state. For example, the information processing system 100 of the embodiment may be used to control a device used for the recovery of physical strength or treatment of decompression sickness, like a broadly known oxygen capsule. In such a case, a change in pressure (partial pressure of oxygen) from a normal state to the achievement of a hyperoxic state is executed on condition that the pressure change allowing condition is satisfied. Of course, the technique of the embodiment can be applied in a pressure change (pressure reduction) in the case of returning from the hyperoxic state to the normal state.

### 5.3 Information Processing Device and Acclimatization Indicator Display Device

The technique of the embodiment can also be applied to an information processing device including: an acquisition unit which acquires biological information of a user present in a closed space; and a processing unit which controls an environmental state of the closed space on the basis of the biological information. The biological information includes at least arterial blood oxygen saturation information. The processing unit controls the environmental state of the closed space at least on the basis of the arterial blood oxygen saturation information.

The information processing device in this example may be the control device 300 in FIG. 3. In this case, the acquisition unit of the information processing device is an interface for acquiring biological information from the wearable device 200, and is a receiving processing unit (communication unit), for example. The processing unit of the information processing device corresponds to the processing unit 130 provided on the control device 300 described above.

As described above, the processing unit of the information processing device performs control to change the environmental state of the closed space if the value of the arterial blood oxygen saturation information is equal to or above a predetermined threshold.

However, the information processing device in this example is not limited to the control device 300 of FIG. 3 and may be another device such as a PC or server system connected via a network, for example. That is, the information processing device which monitors and manages a hypoxic chamber may be provided in a different place where the closed space 60 (hypoxic chamber) is provided. Thus, it is possible to control a plurality of hypoxic chambers by a single information processing device, or the like. Therefore, the business operator having know-how about the management (operation and monitoring) of the hypoxic chambers can install an information processing device, and using this information processing device, the business operator can centrally manage and control multiple hypoxic chambers used by users.

The technique of the embodiment can also be applied to an acclimatization indicator display device including: an acquisition unit which acquires an acclimatization indicator indicating the degree of acclimatization of a user present in the closed space 60 to the environmental state of the closed space 60 on the basis of biological information including at least arterial blood oxygen saturation information; and a display unit which displays the acclimatization indicator.

The acclimatization indicator display device in this example may be the control device 300 shown in FIG. 3 or the information processing device described above. That is, the acclimatization indicator display device may control pressure or concentration and display an acclimatization indicator acquired through the control, to the user in the format shown in FIG. 10 or the like.

However, the acclimatization indicator display device may only have to be able to acquire and display the resulting acclimatization indicator, and does not necessarily have to perform the environmental control on the closed space 60, the calculation of the acclimatization indicator and the like. That is, the acclimatization indicator display device according to the embodiment may be a different device from the control device 300, and specifically, may be implemented by a PC or smartphone used by the user.

The embodiments to which the invention is applied and the modifications of the embodiments are described above. However, the invention is not limited to the respective embodiments and the modifications thereof. In carrying out the invention, its components can be embodied in modified manners without departing from the scope of the invention. Also, by suitably combining a plurality of components disclosed in the respective embodiments and modifications, various inventions can be formed. For example, some of the components described in the respective embodiments and modifications can be deleted. Also, components described in different embodiments and modifications can be suitably combined. Moreover, a term described along with a different term having a broader meaning or the same meaning at least once in the specification or drawings can be replaced with the different term at any point in the specification or drawings. Thus, various modifications and applications are possible without departing from the scope of the invention.

What is claimed is:

1. An information processing system comprising:
a measuring unit which measures biological information of a user present in a closed space; and
a processing unit which controls an environmental state of the closed space on the basis of the biological information;
wherein the processing unit controls a pressure which is an air pressure or partial pressure of oxygen in the closed space, or a concentration which is an oxygen concentration in the closed space, on the basis of the biological information.

2. The information processing system according to claim 1, wherein
the processing unit performs control to change the pressure or the concentration in the closed space if a value of the biological information satisfies a predetermined control execution condition.

3. The information processing system according to claim 2, wherein
the processing unit performs control to change the pressure or the concentration in the closed space if the value of the biological information satisfies the control execution condition and the pressure or the concentration in the closed space does not reach a predetermined set value.

4. The information processing system according to claim 2, wherein
the processing unit performs control to maintain the pressure or the concentration in the closed space if the value of the biological information does not satisfy the control execution condition.

5. The information processing system according to claim 1, wherein
the processing unit finds an acclimatization indicator of the user on the basis of the biological information of the user.

6. The information processing system according to claim 3, wherein
the processing unit finds, as an acclimatization indicator of the user, at least one of pressure information about the pressure in the case where the value of the biological information no longer satisfies the control execution condition, concentration information about the concentration in the case where the value of the biological information no longer satisfies the control execution condition, and time information about a timing when the value of the biological information satisfies the control execution condition after the pressure reaches the set value.

7. The information processing system according to claim 6, wherein
the processing unit finds, as the pressure information which is the acclimatization indicator, at least one of a value of the pressure in the case where the value of the biological information no longer satisfies the control execution condition, and difference information indicating a change in the pressure over a period until the value of the biological information no longer satisfies the control execution condition from before the pressure begins to change.

8. The information processing system according to claim 6, wherein
the processing unit finds, as the concentration information which is the acclimatization indicator, at least one of a value of the concentration in the case where the value of the biological information no longer satisfies the control execution condition, and difference information indicating a change in the concentration over a period until the value of the biological information no longer satisfies the control execution condition from before the concentration begins to change.

9. The information processing system according to claim 7, wherein
the processing unit finds, as the time information which is the acclimatization indicator, at least one of first time information indicating a time period from a timing when the pressure or the concentration begins to change to a timing after the pressure or the concentration reaches the set value and when the value of the biological information satisfies the control execution condition, and second time information indicating a time period from a timing when the pressure or the concentration reaches the set value to a timing when the value of the biological information satisfies the control execution condition.

10. The information processing system according to claim 1, wherein
the processing unit determines whether to return the pressure or the concentration to a reference value or not, on the basis of the biological information in a sleeping state of the user.

11. The information processing system according to claim 3, wherein
the processing unit determines whether to return the pressure or the concentration to a reference value or not, on the basis of the biological information after the pressure or the concentration in the closed space reaches the set value.

12. The information processing system according to claim 10, wherein
the processing unit performs control to return the pressure or the concentration to the reference value, if a number of times the value of the biological information satisfies a predetermined interruption condition reaches a predetermined number of times or above.

13. The information processing system according to claim 1, wherein
the biological information includes arterial blood oxygen saturation information.

14. An information processing system comprising:
a measuring unit which measures biological information of a user present in a closed space; and
a processing unit which controls an environmental state of the closed space on the basis of the biological information;
wherein the biological information includes arterial blood oxygen saturation information, and
the processing unit controls the environmental state of the closed space on the basis of the arterial blood oxygen saturation information.

15. The information processing system according to claim 14, wherein
the processing unit performs control to change the environmental state of the closed space on condition that a value of the arterial blood oxygen saturation information is equal to or above a predetermined threshold.

16. An information processing device comprising:
an acquisition unit which acquires biological information of a user present in a closed space; and
a processing unit which controls an environmental state of the closed space on the basis of the biological information;
wherein the biological information includes at least arterial blood oxygen saturation information, and
the processing unit controls the environmental state of the closed space, at least on the basis of the arterial blood oxygen saturation information.

17. The information processing device according to claim 16, wherein
the processing unit performs control to change the environmental state of the closed space if a value of the arterial blood oxygen saturation information is equal to or above a predetermined threshold.

18. An acclimatization indicator display device comprising:
an acquisition unit which acquires an acclimatization indicator indicating an extent of acclimatization of a user present in a closed space to an environmental state of the closed space on the basis of biological information including at least arterial blood oxygen saturation information of the user; and
a display unit which displays the acclimatization indicator.

19. A method for controlling an information processing system comprising:
performing measurement processing of biological information of a user present in a closed space; and
controlling a pressure which is an air pressure or partial pressure of oxygen in the closed space, or a concentration which is an oxygen concentration in the closed space, on the basis of the biological information.

20. A method for controlling an information processing system comprising:
performing measurement processing of biological information including at least arterial blood oxygen saturation information of a user present in a closed space; and
controlling an environmental state of the closed space on the basis of the arterial blood oxygen saturation information.
